(19) United States
(12) Patent Application Publication
(10) Pub. No.: US 2026/0131075 A1
(43) Pub. Date: May 14, 2026

(54) SYSTEMS AND METHODS FOR MANUFACTURING INFUSION CANNULAS

(71) Applicant: Tandem Diabetes Care, Inc, San Diego, CA (US)

(72) Inventors: Justin Coker, San Diego, CA (US); Kenneth Hsu, San Diego, CA (US); Steven Reynolds, San Diego, CA (US)

(21) Appl. No.: 19/379,004

(22) Filed: Nov. 4, 2025

Related U.S. Application Data
(60) Provisional application No. 63/719,283, filed on Nov. 12, 2024.

(51) Int. Cl. A61M 5/158

(52) U.S. Cl. CPC A61M 5/158; A61M 2207/10

(57) ABSTRACT

An alternative mechanism for forming a cannula assembly having an internal coil does not require a separate tipping process for forming a cannula tip or leaving a length of tubing extending beyond the coil for formation of the tip when forming each catheter. The injection molding process that is used to form the cannula hub on the proximal end of the cannula can be modified to balance the mold flow and runners used in the process to simultaneously mold a tapered tip onto a distal end of the cannula at the same time that the infusion hub is molded.

### PRIORITY CLAIM

The present application claims the benefit of U.S. Provisional Application No. 63/719,283 filed November 12, 2024, which is hereby incorporated herein by reference in its entirety.

### TECHNICAL FIELD

The present disclosure relates to manufacturing of infusion sets that facilitate injection of medicament into a body of a patient.

### BACKGROUND

There are a wide variety of medical treatments that include the administration of a therapeutic fluid in precise, known amounts at predetermined intervals. Devices and methods that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Patent Nos. 8,287,495; 8,573,027; 8,986,253; and 9,381,297, each of which is incorporated herein by reference in its entirety.

Some portable infusion pumps deliver medicament to patients through infusion sets that include tubing extending from the pump and a cannula with an associated needle positioned transcutaneously (i.e., through the patient’s skin) at an infusion site to allow infusion of the medicament through a cannula and into the patient. Such pumps can be worn on the body or carried near the body (e.g., in the user’s pocket) with the infusion site situated on the patient’s body and connected with the pump via the tubing. Other pumps that are worn directly on the body can deliver medicament through a cannula that extends directly beneath the pump.

If a patient leaves the cannula injected at the infusion site at one location for too long a period of time, or uses the same infusion site repeatedly, unwanted side effects such as infection and the accumulation of fat and scar tissue can result. Therefore, patients are often instructed to rotate infusion sites to avoid or minimize side effects. Depending on the type of cannula used, the general physiological response of the patient with regard to insulin absorption, and other factors, the time needed between infusion site rotations can vary. Commonly, sites are rotated every 24-48 hours or every 48-72 hours and extended wear infusion sets that can be worn for a longer period of time, such as for example, 7 days, are also being developed.

Embodiments of various infusion sets are described in U.S. Patent Publication Nos. 2018/0280608, 2021/0402084, 2022/0226568 and 2023/0277765, each of which is hereby incorporated herein by reference in its entirety. Some of these infusion sets include a coil extending through the delivery cannula inserted into the user for delivery of medicament into the user’s skin. Use of such a coil within the cannula can provide a number of benefits to the user including, but not limited to, flexibility and kink resistance, which increase the wear time of an infusion set. The coil can also provide a filtering function to capture and retain aggregate particles formed in the liquid medication. Such aggregate particles can induce or contribute to infusion site inflammatory and immune responses if delivered and can compromise the infusion site.

While cannulas with internal coils provide significant advantages, there are technical challenges and increased costs associated with the manufacturing process. For example, the coils must be manually inserted on a long mandrel, the coils must be manually spaced and aligned along the length of the mandrel, care must be taken so that the coils do not shift during the extrusion process, the mandrel must be removed from the extruded coils, and the extruded coils must be cut to size. These requirements, alone or in combination with one another, can be a challenge for large scale manufacturing. Additional steps are also be taken to prepare the cannulas for use, including forming a tip on a distal end of the cannula in a cannula tipping process and injection molding a cannula hub on proximal end of the cannula.

### SUMMARY

The present disclosure providing an alternative mechanism for forming a cannula assembly that does not require a separate tipping process for forming a cannula tip or leaving a length of tubing extending beyond the coil for formation of the tip when forming each catheter. The injection molding process that is used to form the cannula hub on the proximal end of the cannula can be modified to balance the mold flow and runners used in the process to simultaneously mold a tapered tip onto a distal end of the cannula at the same time that the infusion hub is molded.

In an embodiment, a method of manufacturing a plurality of cannulas configured to facilitate delivery of medicament to a user includes arranging an elongate coil on a mandrel and processing the mandrel and elongate coil through an extrusion device to form an elongate cannula body with an outer tube around the mandrel and the elongate coil. The elongate cannula body can then be cut to create a plurality of cannula body portions. A tapered tip can then be formed on a distal end and a cannula hub on a proximal end of each cannula body portion in an injection molding process.

In an embodiment, a cannula configured to facilitate delivery of medicament to a user is formed by a process that includes arranging an elongate coil on a mandrel and processing the mandrel and elongate coil through an extrusion device to form an elongate cannula body with an outer tube around the mandrel and the elongate coil. The elongate cannula body can be to create a plurality of cannula body portions. An injection molding process can be used to form a tapered tip on a distal end and a cannula hub on a proximal end of each cannula body portion.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an ambulatory infusion pump with an infusion set according to an embodiment of the disclosure.

FIGS. 2A-2C depict an ambulatory infusion pump according to an embodiment of the disclosure.

FIG. 3 depicts an infusion set according to an embodiment of the disclosure.

FIGS. 4A-4E depict a cannula according to an embodiment of the disclosure.

FIGS. 5-5C depict an assembly used in creating a plurality of cannulas according to an embodiment of the disclosure.

FIGS. 6A-6C depict schematic representations of aspects of an injection molding arrangement to manufacture a cannula assembly according to the disclosure.

FIG. 7 depicts a flowchart of steps in a method of manufacturing a plurality of cannula assemblies according to the disclosure.

FIG. 8 depicts a cannula assembly according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

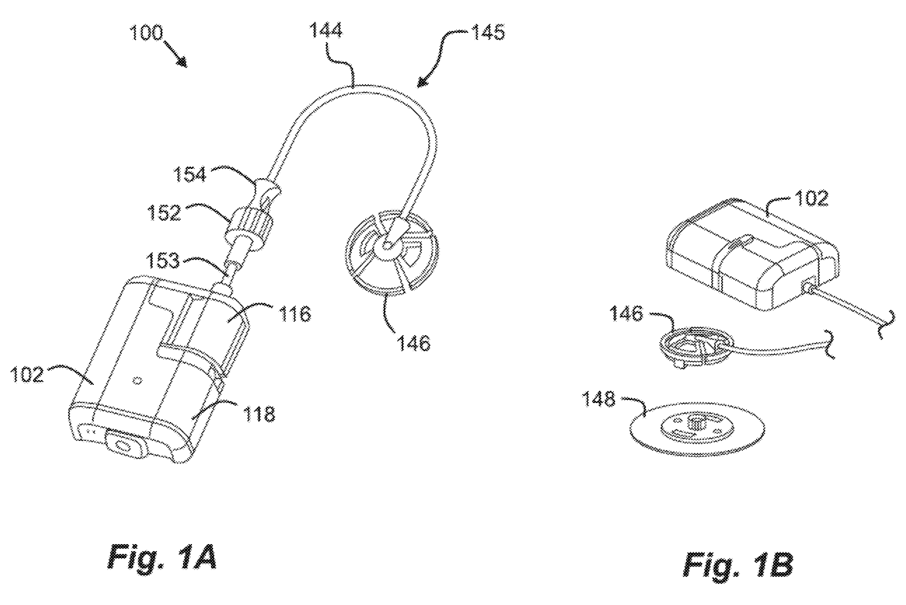

FIGS. 1A-1B depict an exemplary medical device that can be used with embodiments of the disclosure. In this embodiment, the medical device is configured as a pump 102, such as an infusion pump, that can include a pumping or delivery mechanism and a reservoir for delivering a medicament to a patient. In one embodiment, the medical device can be a portable pump configured to deliver insulin to a patient. Further details regarding such pump devices can be found in U.S. Patent Nos. 8,287,495, 10,279,107 and 10,864,318, each of which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion hub 148 at an infusion site on a user through which medicament from the pump is delivered to a patient through a cannula extending from the infusion hub 148 into the user’s skin.

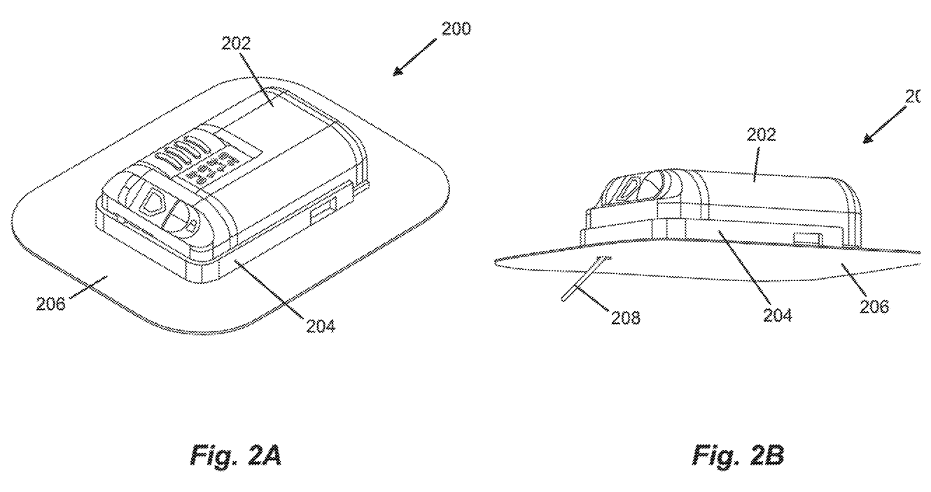

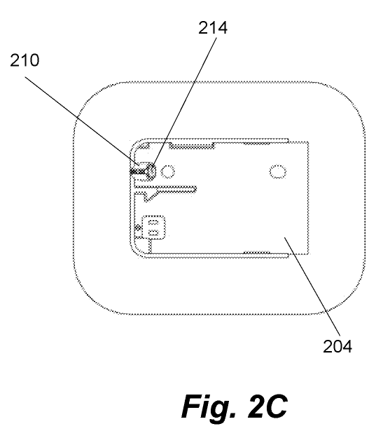

FIGS. 2A-2C depict an infusion pump system 200 according to another embodiment of the disclosure. System 200 can include an infusion pump 202 configured as a patch pump that is worn directly on the body of the user with an adhesive patch 206. Pump 202 can be affixed to the body of the user with an adhesive patch 206 carrying a tray 204 thereon that releasably attaches the pump 202 to the tray 204. Pump 202 can be configured to deliver medicament to the user through a cannula 208 extending directly beneath the pump 202 and adhesive patch 206. Referring to FIG. 2C, in embodiments tray 204 can include a cannula port 210 through which cannula 208 is inserted. The cannula port 210 can contain a septum 214 therein that facilitates transfer of insulin from the pump 202 to the cannula 208.

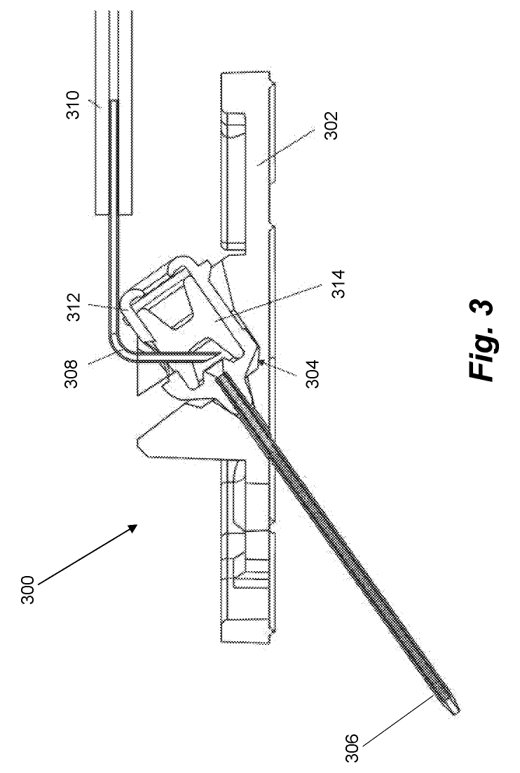

FIG. 3 depicts an infusion hub 300 of an infusion set according to an embodiment of the disclosure. Infusion hub 300 can include a patch 302 or other mechanism configured to adhere to the patient and a cannula hub or barrel 304 connected to a cannula 306. A fluid introducer needle 308 can interface tubing 310 with the infusion hub 300. The barrel 304 can include a mechanical housing 312 configured to house a septum 314. A fluid path for medication does not exist when the cannula 306 is positioned within the cannula hub or barrel 304 because the septum 314 has no opening when the cannula is positioned within the hub or barrel 304. The fluid introducer needle 308 can be configured to pierce the septum 314 to deliver fluid from the tubing 310 to the cannula 306. Thus, the fluid path for the insulin medication, therefore, can be created by introduction of the fluid introducer needle 308. The fluid path created by the fluid introducer needle 308 can run from a reservoir of a pump through infusion tubing extending from the pump, through one or more connectors connecting the infusion tubing of the pump with tubing 310 of an infusion set, through the tubing 310 of the infusion set, to the barrel 304 and to the patient via the cannula 306. Although infusion hub 300 is depicted as being employed with a length of tubing 310 that may interface with tubing extending from a pump such as depicted in FIGS. 1A-1B, the components of infusion hub 300 such as the septum 314, cannula 306, etc. can be adapted to be incorporated into a patch pump system such as the system depicted in FIGS. 2A-2B.

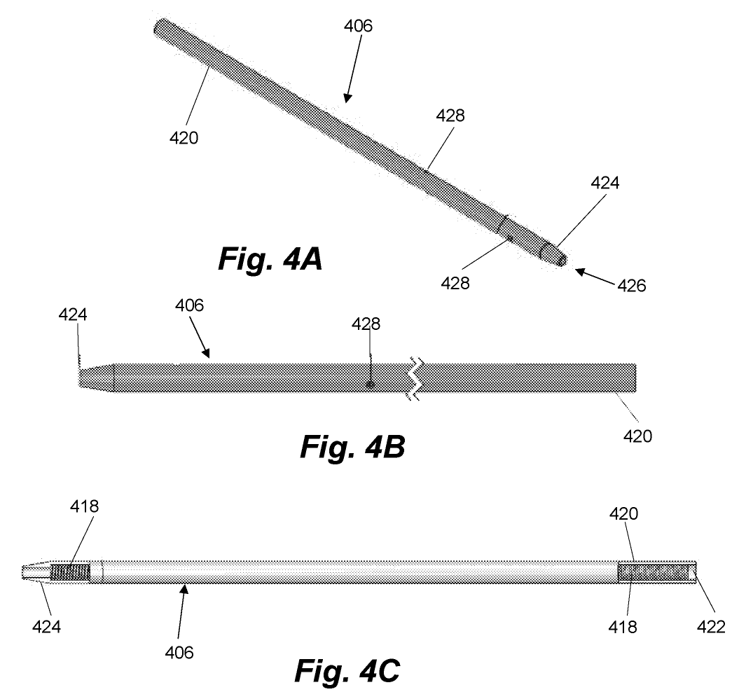

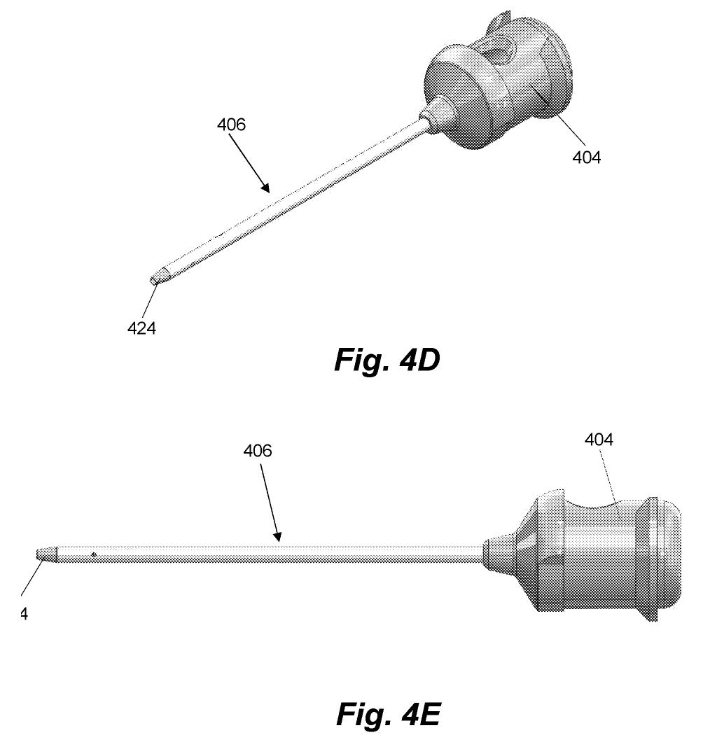

FIGS. 4A-4C depict a cannula 406 for use in an infusion set, with a patch pump, or to otherwise deliver a liquid medicament into a user according to an aspect of the disclosure. Cannula 406 includes a tube wall 420 having an inner lumen 422 through which medicament can be delivered to a user. A coil 418 can be disposed within inner lumen 422 of cannula 406. Tube wall 420 may comprise a softer, more flexible polymer material, such as, for example, Pebax® and coil 422 can comprise a harder, more rigid material such as, for example, stainless steel. Cannula 406 can further include a tapered distal end 424 (through which the coil does not extend) having a distal opening 426 and one or more infusion holes 428 along the tube wall. The tapered distal end 424 can be a continuation of the tube wall 420 (e.g., a seamless continuation of the same material) with a tapered geometry. For example, the tapered distal end 424 and the tube wall 420 can be formed simultaneously as a single continuous and monolithic construct. In another example, the tapered distal end 424 can be overmolded onto a distal end of the tube wall 420. Forming the tapered distal end 424 and the tube wall 420 together is a single continuous object (either simultaneously or through overmolding) can increase durability and provide more seamless transitions for the cannula 406. Once each cannula 406 has been manufactured the infusion hub 404 can be injection molded onto the cannula to provide the finished cannula assembly for use in an infusion set as shown in FIGS. 4D and 4E.

As noted above, providing a coil inside of a cannula has been found to provide a number of benefits. In some examples, the coil is comprised of a stainless-steel material that is co-extruded to have a polymer sleeve over its external surface. While such configurations provide the advantages noted herein, there are technical challenges and increased costs associated with the manufacturing process. For example, inserting coils on a long mandrel, indexing, keeping the coils from shifting during the extrusion process, mandrel removal, and cutting the extruded coil can be a challenge for large scale manufacturing.

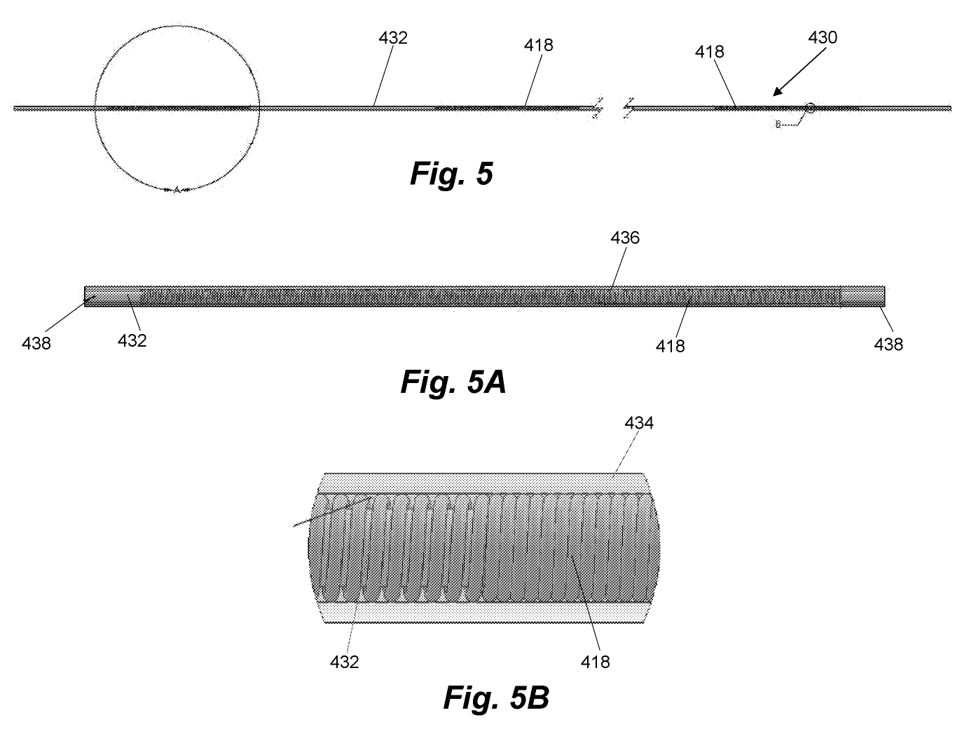

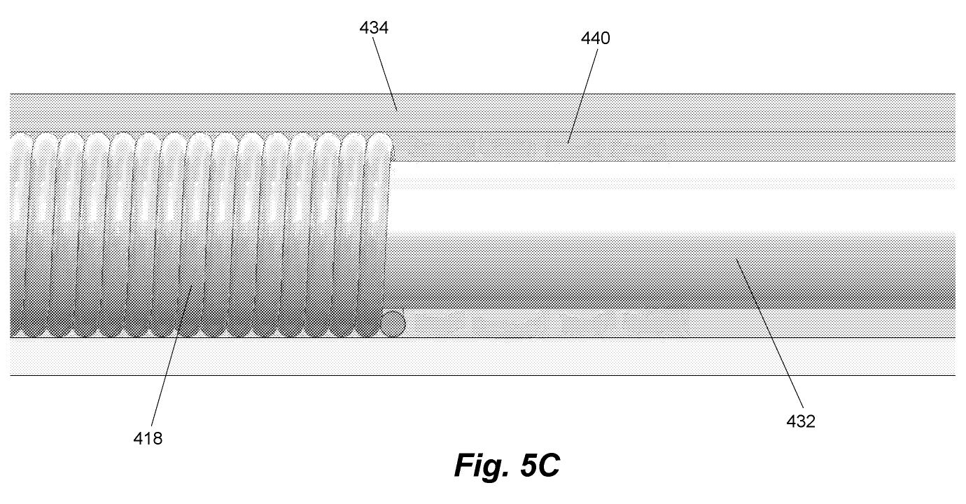

One method of manufacturing cannulas with an internal coil as described above includes arranging a plurality of coils on a mandrel that may be comprised of, for example, stainless steel. The mandrel and coils are then fed through an extrusion machine that provides a polymer (e.g., Pebax®) overcoat around the coils and mandrel. FIGS. 5A-5C depict an assembly 430 at this stage of the process. A plurality of coils 418 are positioned on a mandrel 432 in a spaced apart fashion, such as, for example, 1 inch apart. A tubular wall 434 provided in the extruder surrounds the coils 418 and mandrel 432. The assembly 430 would then be processed into a plurality (e.g., 10) sections 436 by cutting the assembly between the coils (the mandrel can be removed before or after cutting the sections). As depicted in FIG. 5A, each section 436 includes a length of tubing 438 on each end that extends beyond the coil 418. Each cut section 436 can be processed into a cannula by providing the tapered distal end 424 and infusion holes 428 (See FIGS. 4A-4C).

Referring to FIG. 5C, there is an open space 440 between each coil on the mandrel 432. The primary purpose of this open space is to provide a section of tubing without a coil that can be processed into the tapered distal tip 424 for each cannula. It has been found that it is difficult to maintain the spacing of the coils 418 when assembling the coils 418 on the mandrel 432 and that the forces applied by the extrusion machine can also cause the coils 418 to move on the mandrel 432. This can result in coils 418 that are too close together within the tubular wall 434 of the assembly 430 to provide a sufficient length of tubing 438 extending beyond the coils 418 for formation of the tapered distal end 434 of the cannula. In addition, the process of forming the tapered distal tip 424 has been found to require significant manual effort and attempts to automate the tipping process for high volume manufacturing have been found to yield less than desirable results.

The present disclosure solves the above issues by providing an alternative mechanism for forming the cannula tip that does not require a separate tipping process or leaving a length of tubing extending beyond the coil for formation of the tip when forming each catheter. As noted above, the manufacturing process for the cannula assembly already includes an injection molding process for forming the infusion hub onto the cannula. This process can be modified to balance the mold flow and runners used in the process to simultaneously mold a tapered tip onto a distal end of the cannula when the infusion hub is molded onto the proximal end of the cannula. This eliminates the separate process for forming the catheter tip without adding any additional manufacturing steps. In addition, without the need for the extra length of tubing extending beyond the coil for formation of the tip, the coiled cannula sections can be formed with a single continuous coil that is cut into individual coiled cannula sections onto which the tapered tip and infusion hub are injection molded, eliminating the complications caused by separate spaced apart coils described above.

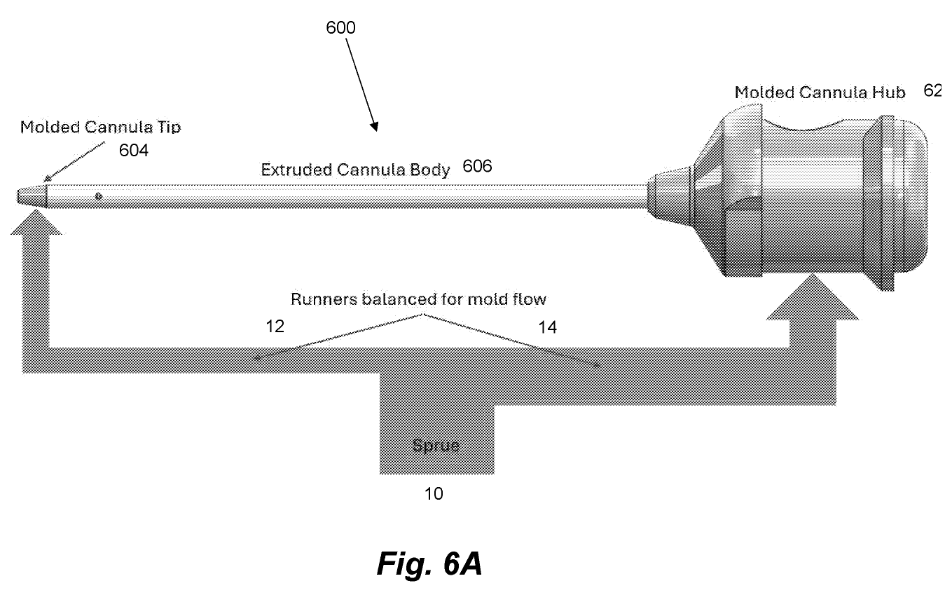

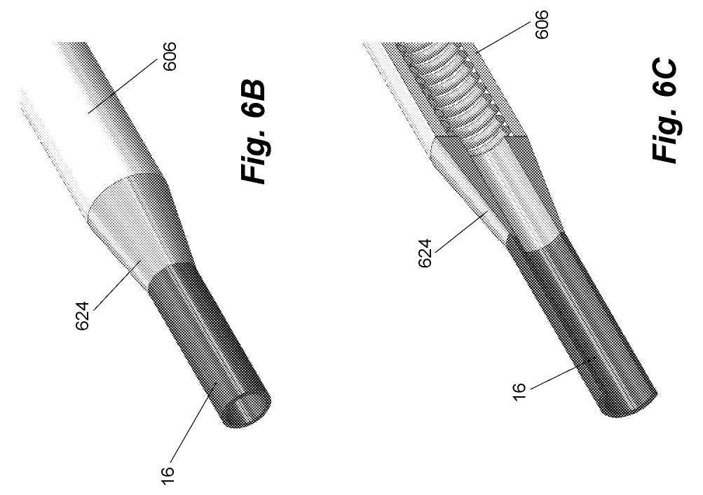

Referring now to FIG. 6A, a schematic representation of one example embodiment of an injection molding arrangement to manufacture a cannula assembly 600 according to the present disclosure is depicted. Cannula body 606 has previously been manufactured with an internal coil as described above with a tubular wall having been extruded over a single long coil and multiple identical cannula bodies 606 being cut to size from the tubular wall. Cannula body 606 is then arranged in an injection molding machine. Material is introduced into sprue 10 and flows through runners 12, 14 that split towards molds at opposite ends of the cannula body 606 for creation of the molded cannula tip 624 and infusion hub 604, respectively. Due to the greater amount of material needed to form infusion hub 604, that runner 14 is wider than runner 12 extending to cannula tip 624 to balance the flow and generally provide for both components to be formed simultaneously. In embodiments, a cylindrical fan gate 16 as shown in FIGS. 6B-6C can be employed in the injection molding process to deliver the material to form the cannula tip 624 on the distal end of the cannula body 606 into the mold. Although fan gate 16 is depicted as oriented towards a front of the cannula tip 624, fan gate 16 could be oriented in other directions (e.g., from the side).

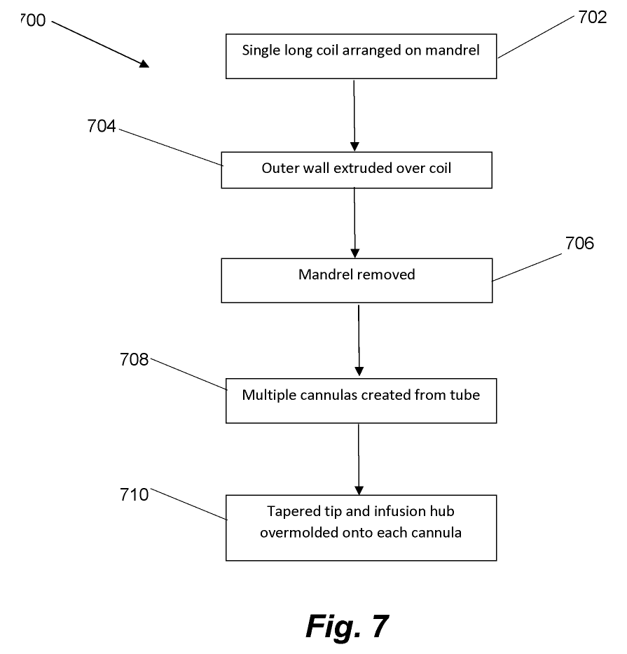

FIG. 7 depicts a flowchart of steps in a method of manufacturing a plurality of cannulas 700 for use in an infusion set, patch pump, or other infusion mechanism according to an embodiment of the disclosure. At step 702, a single elongate coil is arranged on a mandrel. This assembly is processed through an extrusion device at step 704 to form an outer tubular wall over the elongate coil. Following extrusion, the mandrel can be removed at step 706 leaving an elongate tube having a continuous internal coil along the entire length of the tube. The tube can then be cut to length to form multiple cannula bodies with internal coils at step 708. At step 710 both the tapered tip and the infusion hub can be overmolded onto the cannula body in an injection molding process as described above. Additional processing steps may also be performed to prepare the cannula for a particular use. For example, infusion holes can be formed in a side of the cannula body. If a laser is user to cut the elongate tube into multiple cannula bodies at step 706, it may be advantageous to also use the laser to form the infusion holes at that time.

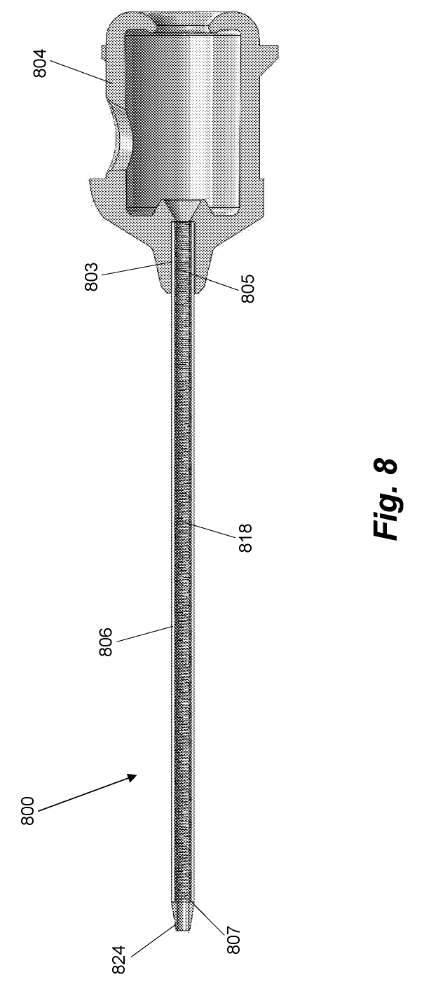

FIG. 8 depicts an example embodiment of a cannula assembly 800 manufactured according to the present disclosure. Cannula body 806 includes an internal coil 818. Tapered distal tip 824 has been overmolded onto a distal end 807 of cannula body 806. Cannula hub 804 is molded onto a proximal end of cannula body 806 such that there is an overlap between cannula hub 804 and cannula body 806. In particular, a proximal portion 805 of cannula body nests within a recess 803 in cannula hub 804. This provides additional contact surface area for a stronger adhesive bond between the components to prevent the cannula hub 804 from detaching from the cannula body.

In some embodiments, materials having different durometers can be used to form the cannula body and the cannula hub. For example, in one embodiment, the cannula body can be formed from 72/33 Pebax® and the cannula hub from 63/33 Pebax®. This can create a stronger chemical bond providing better adhesion between the cannula hub and the cannula body. The cannula tip can also be the same or different as the cannula body and/or cannula hub.

The systems and methods described herein therefore simplify the upstream manufacturing process of the plurality of cannulas. There is also likely cost savings in eliminating the discrete short length coils for the single long length coil as material handling is simplified. There is also likely cost savings in eliminating the separate catheter tip forming process to generate the tapered tip. The elimination of this process step may make the cannula manufacturing process easier to scale up for high volume production.

In an embodiment, a method of manufacturing a plurality of cannulas configured to facilitate delivery of medicament to a user includes arranging an elongate coil on a mandrel and processing the mandrel and elongate coil through an extrusion device to form an elongate cannula body with an outer tube around the mandrel and the elongate coil. The elongate cannula body can then be cut to create a plurality of cannula body portions. A tapered tip can then be formed on a distal end and a cannula hub on a proximal end of each cannula body portion in an injection molding process.

In embodiments, the injection molding process for each cannula body portion can include arranging the cannula body portion in an injection molding machine and introducing material into a sprue such that material flows through a first runner that splits towards a first mold at a proximal end of the cannula body for creation of the cannula hub and a second runner that splits towards a second mold at a distal end of the cannula body for creation of the tapered tip.

In embodiments, the first runner that directs material towards the first mold at the proximal end is wider than the second runner that directs material towards the distal end

In embodiments, the injection molding process further includes providing a fan gate adjacent the distal end of the cannula body to deliver the material to form the cannula tip.

In embodiments, the tapered tip and the cannula hub are formed generally simultaneously

In embodiments, the cannula hub is molded onto the cannula body such that a portion of the proximal end of the cannula body nests within the cannula hub

In embodiments, a material used to form the cannula hub has a different durometer from a material used to form the cannula body

In embodiments, the mandrel is removed prior to cutting the elongate cannula body

In embodiments, the outer tube comprises a polymer and the coil comprises a metal.

In embodiments, the elongate coil is a single continuous coil.

In an embodiment, a cannula configured to facilitate delivery of medicament to a user is formed by a process that includes arranging an elongate coil on a mandrel and processing the mandrel and elongate coil through an extrusion device to form an elongate cannula body with an outer tube around the mandrel and the elongate coil. The elongate cannula body can be to create a plurality of cannula body portions. An injection molding process can be used to form a tapered tip on a distal end and a cannula hub on a proximal end of each cannula body portion.

In embodiments, the injection molding process for forming a cannula from each cannula body portion can include arranging the cannula body portion in an injection molding machine and introducing material into a sprue such that material flows through a first runner that splits towards a first mold at a proximal end of the cannula body for creation of the cannula hub and a second runner that splits towards a second mold at a distal end of the cannula body for creation of the tapered tip.

In embodiments, the first runner that directs material towards the first mold at the proximal end is wider than the second runner that directs material towards the distal end

In embodiments, the injection molding process further includes providing a fan gate adjacent the distal end of the cannula body to deliver the material to form the cannula tip.

In embodiments, the tapered tip and the cannula hub are formed generally simultaneously

In embodiments, the cannula hub is molded onto the cannula body such that a portion of the proximal end of the cannula body nests within the cannula hub

In embodiments, a material used to form the cannula hub has a different durometer from a material used to form the cannula body

In embodiments, the process further comprises removing the mandrel prior to cutting the elongate cannula body

In embodiments, the outer tube comprises a polymer and the coil comprises a metal.

In embodiments, the elongate coil is a single continuous coil.

In embodiments, a system for manufacturing a plurality of cannulas as described herein is provided.

In embodiments, an infusion set incorporating a cannula as disclosed herein is provided.

In embodiments, an ambulatory infusion pump system incorporating a cannula as disclosed herein is provided.

Although the infusion pump embodiments herein are specifically described primarily with respect to the delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

Also incorporated herein by reference in their entirety are commonly owned U.S. Patent Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; 12,214,159; and 12,357,751 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. Patent Applications Nos. 17/368,968; 17/896,492; 18/398,543; 18/962,169; 19/003,140; 19/003,164; 19/119,554; 19/134,333; 19/205,083; 19/220,426; 19/221,933; 19/225,150; and 19/252,256.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A method of manufacturing a plurality of cannulas configured to facilitate delivery of medicament to a user, comprising:
arranging an elongate coil on a mandrel;
processing the mandrel and elongate coil through an extrusion device to form an elongate cannula body with an outer tube around the mandrel and the elongate coil;
cutting the elongate cannula body to create a plurality of cannula body portions; and
forming a tapered tip on a distal end and a cannula hub on a proximal end of each cannula body portion in an injection molding process.

2. The method of claim 1, wherein the injection molding process for each cannula body portion comprises:
arranging the cannula body portion in an injection molding machine; and
introducing material into a sprue such that material flows through a first runner that splits towards a first mold at a proximal end of the cannula body for creation of the cannula hub and a second runner that splits towards a second mold at a distal end of the cannula body for creation of the tapered tip.

3. The method of claim 2, wherein the first runner that directs material towards the first mold at the proximal end is wider than the second runner that directs material towards the distal end.

4. The method of claim 2, wherein the injection molding process further includes providing a fan gate adjacent the distal end of the cannula body to deliver the material to form the cannula tip.

5. The method of claim 1, wherein the tapered tip and the cannula hub are formed generally simultaneously.

6. The method of claim 1, wherein the cannula hub is molded onto the cannula body such that a portion of the proximal end of the cannula body nests within the cannula hub.

7. The method of claim 1, wherein a material used to form the cannula hub has a different durometer from a material used to form the cannula body.

8. The method of claim 1, further comprising removing the mandrel prior to cutting the elongate cannula body.

9. The method of claim 1, wherein the outer tube comprises a polymer and the coil comprises a metal.

10. The method of claim 1, where the elongate coil is a single continuous coil.

11. A cannula configured to facilitate delivery of medicament to a user formed by a process comprising:
arranging an elongate coil on a mandrel;
processing the mandrel and elongate coil through an extrusion device to form an elongate cannula body with an outer tube around the mandrel and the elongate coil;
cutting the elongate cannula body to create a plurality of cannula body portions; and
forming a tapered tip on a distal end and a cannula hub on a proximal end of each cannula body portion in an injection molding process.

12. The cannula of claim 1, wherein the injection molding process for forming a cannula from each cannula body portion comprises:
arranging the cannula body portion in an injection molding machine; and
introducing material into a sprue such that material flows through a first runner that splits towards a first mold at a proximal end of the cannula body for creation of the cannula hub and a second runner that splits towards a second mold at a distal end of the cannula body for creation of the tapered tip.

13. The cannula of claim 12, wherein the first runner that directs material towards the first mold at the proximal end is wider than the second runner that directs material towards the distal end.

14. The cannula of claim 12, wherein the injection molding process further includes providing a fan gate adjacent the distal end of the cannula body to deliver the material to form the cannula tip.

15. The cannula of claim 11, wherein the tapered tip and the cannula hub are formed generally simultaneously.

16. The cannula of claim 11, wherein the cannula hub is molded onto the cannula body such that a portion of the proximal end of the cannula body nests within the cannula hub.

17. The cannula of claim 11, wherein a material used to form the cannula hub has a different durometer from a material used to form the cannula body.

18. The cannula of claim 11, wherein the process further comprises removing the mandrel prior to cutting the elongate cannula body.

19. The cannula of claim 11, wherein the outer tube comprises a polymer and the coil comprises a metal.

20. The cannula of claim 11, where the elongate coil is a single continuous coil.
